(19) United States
(12) Patent Application Publication
(10) Pub. No.: US 2024/0382641 A1
(43) Pub. Date: Nov. 21, 2024

(54) WEARABLE ELECTRONIC DEVICE FOR PROVIDING OR AFFECTING SMELL

(71) Applicant: City University of Hong Kong, Kowloon (HK)

(72) Inventors: Xinge Yu, Kowloon (HK); Yiming Liu, Kowloon (HK); Yanli Jiao, Kowloon (HK); Wooyoung Park, Kowloon (HK)

(21) Appl. No.: 18/320,495

(22) Filed: May 19, 2023

(51) Int. Cl. A61L 9/03

(52) U.S. Cl. CPC A61L 9/035; A61L 2209/111; A61L 2209/15

(57) ABSTRACT

A wearable electronic device for providing or affecting smell. The wearable electronic device includes one or more odor manipulation units and a control circuit arrangement operably coupled with the one or more odor manipulation units for operating the one or more odor manipulation units. Each of the one or more odor manipulation units is respectively operable to provide or affect smell perceivable by a user of the wearable electronic device.

### TECHNICAL FIELD

The invention relates to a wearable electronic device for providing or affecting smell. The wearable electronic device may be suitable for use by user with sensory impairment such as user with vision and/or hearing loss.

### BACKGROUND

Electronic devices for providing or affecting smell are known. Typically, these electronic devices can generate and provide odor to an environment in which a subject is present or to a subject. The odor may be provided by odorant included in a phase change medium (phase change material(s)) carried by the electronic device. When the phase change medium dissolves or vaporizes, the odorant can be released, volatilized, or diffused into the environment. The odor provided by the electronic device can be processed by an olfactory organ of the subject to identify an associated smell. Problems associated with some of these electronic devices include, e.g., some of these electronic devices are bulky and rigid hence not suitable for some applications, some of these electronic devices can only provide simple smell or limited number of smell (e.g., a single smell), which is not suitable or not sufficient for some applications, etc.

### SUMMARY OF THE INVENTION

In a first aspect, there is provided a wearable electronic device for providing or affecting smell, comprising: one or more odor manipulation units each respectively operable to provide or affect smell perceivable by a user of the wearable electronic device; and a control circuit arrangement operably coupled with the one or more odor manipulation units for operating the one or more odor manipulation units. In some embodiments, the one or more odor manipulation units may include one or more odor generation units. In some embodiments in which the wearable electronic device includes multiple odor manipulation units, different odor manipulation units may be arranged to provide or affect different smell (e.g., different spatial and/or temporal odor profiles). In some embodiments in which the wearable electronic device includes multiple odor manipulation units, different odor manipulation units may be arranged to provide or affect the same smell (e.g., the same spatial and/or temporal odor profile).

Optionally, the wearable electronic device further comprises a substrate. In some embodiments, the substrate may be flexible and/or elastomeric. In some embodiments, the substrate may be made of plastic material(s). For example, the substrate may be made of polydimethylsiloxane (PDMS), soft silicon elastomer such as Ecoflex™, silicone rubber, etc. For example, the substrate may be made of hydrogel. The substrate may include one or more substrate layers.

Optionally, the one or more odor manipulation units and/or the control circuit arrangement are supported by or mounted to the substrate. In some embodiments, the substrate may include, at least, an upper substrate layer and a lower substrate layer, and the control circuit arrangement may be arranged between the upper substrate layer and the lower substrate layer. In some embodiments, the upper substrate layer and/or the lower substrate layer may include cut-out(s) or opening(s) through which at least part of one or more odor manipulation units could at least partly extend.

Optionally, the substrate is constructed (e.g., shaped and/or sized and/or made of suitable material(s)) to enable or facilitate wearing of the wearable electronic device on a face of the user. Optionally, the substrate is constructed (e.g., shaped and/or sized and/or made of suitable material(s)) to enable or facilitate wearing of the wearable electronic device on or near a nose of the user. In some embodiments, the substrate is constructed (e.g., shaped and/or sized and/or made of suitable material(s)) to enable or facilitate wearing at least part of the wearable electronic device between a nose and a mouth (e.g., upper lip) of the user.

Optionally, the one or more odor manipulation units comprises: a first odor manipulation unit configured (e.g., shaped and/or sized and/or supported by or mounted to the substrate in such a way) to be placed closer to one nostril of the user when the wearable electronic device is worn on or near the nose of the user, and a second odor manipulation unit configured (e.g., shaped and/or sized and/or supported by or mounted to the substrate in such a way) to be placed closer to another nostril of the user when the wearable electronic device is worn on or near the nose of the user. The one or more odor manipulation units may or may not include further odor manipulation unit(s). In some embodiments, the one or more odor manipulation units may include one or more additional odor manipulation units (in addition to the first and second odor manipulation units). In some embodiments, the one or more odor manipulation units consist only of the first and second (i.e., only two) odor manipulation units.

Optionally, the control circuit arrangement is arranged to independently operate the first odor manipulation unit and the second odor manipulation unit. In some embodiments, the control circuit arrangement may be arranged to simultaneously operate the first and second odor manipulation units. In some embodiments, the control circuit arrangement may be arranged to selectively operate the first and second odor manipulation units.

Optionally, each of the one or more odor manipulation units respectively comprises two or more odor manipulators. In some embodiments, the odor manipulators may include odor generators.

Optionally, the first odor manipulation unit comprises two or more odor manipulators. Optionally, each of the two or more odor manipulators of the first odor manipulation unit is respectively arranged to provide a respective odor. In some embodiments, each of the two or more odor manipulators of the first odor manipulation unit is respectively arranged to provide a respective type of odor (e.g., primary odor) such that the odor provided by each of the two or more odor manipulators of the first odor manipulation unit are different.

Optionally, the second odor manipulation unit comprises two or more odor manipulators. Optionally, each of the two or more odor manipulators of the second odor manipulation unit is respectively arranged to provide a respective odor. In some embodiments, each of the two or more odor manipulators of the second odor manipulation unit is respectively arranged to provide a respective type of odor (e.g., primary odor) such that the odor provided by each of the two or more odor manipulators of the first odor manipulation unit are different.

Optionally, control circuit arrangement is arranged to operate the two or more odor manipulators of the first odor manipulation unit, simultaneously or selectively. Optionally, control circuit arrangement is arranged to independently operate the two or more odor manipulators of the first odor manipulation unit. In some examples, control circuit arrangement is arranged to operate the two or more odor manipulators of the first odor manipulation unit to provide mono olfaction. In some examples, control circuit arrangement is arranged to operate the two or more odor manipulators of the first odor manipulation unit to provide stereo olfaction. Optionally, control circuit arrangement is arranged to control (e.g., timing, duration, and/or extent of) activation and/or deactivation of each of the two or more odor manipulators of the first odor manipulation unit to affect spatial and/or temporal odor profile (e.g., type(s), timing, intensity, and/or duration of odor) provided by the first odor manipulation unit.

Optionally, control circuit arrangement is arranged to operate the two or more odor manipulators of the second odor manipulation unit, simultaneously or selectively. Optionally, control circuit arrangement is arranged to independently operate the two or more odor manipulators of second odor manipulation unit. In some examples, control circuit arrangement is arranged to operate the two or more odor manipulators of the second odor manipulation unit to provide mono olfaction. In some examples, control circuit arrangement is arranged to operate the two or more odor manipulators of the second odor manipulation unit to provide stereo olfaction. Optionally, control circuit arrangement is arranged to control (e.g., timing, duration, and/or extent of) activation and/or deactivation of each of the two or more odor manipulators of the second odor manipulation unit to affect spatial and/or temporal odor profile (e.g., type(s), timing, intensity, and/or duration of odor) provided by the second odor manipulation unit.

Optionally, control circuit arrangement comprises one or more processors and memory storing one or more sets of control instructions each arranged to be operated by the one or more processors to control respective (e.g., timing, duration, and/or extent of) activation and/or deactivation of: one or more of the odor manipulators of the first odor manipulation unit and/or one or more of the odor manipulators of the second odor manipulation unit. Each of the one or more sets of control instructions may be associated with a respective message recognizable by the user. In some embodiments, each of the one or more processors may include one or more: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), or any other digital or analog circuitry/circuitries configured to interpret and/or to execute program instructions and/or to process signals and/or information and/or data. In some embodiments, the memory may include: one or more volatile memory (such as RAM, DRAM, SRAM, etc.), one or more non-volatile memory (such as ROM, PROM, EPROM, EEPROM, FRAM, MRAM, FLASH, SSD, NAND, NVDIMM, etc.), or any of their combinations.

Optionally, the control circuit arrangement further comprises a power module for wirelessly receiving power for operating the wearable electronic device. The power module may be an inductive power module with one or more inductive coils. The power module may also include energy storage element(s) (e.g., capacitive element(s) such as capacitor(s)).

Optionally, the control circuit arrangement further comprises a communication module for wirelessly communicating data and/or instructions with an external electronic device. The communication module may include one or more of: a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, above 5G, or the like) transceiver, an optical port, an infrared port, a USB connection interface, etc.

Optionally, the power module and the communication module may be integrated as a single module. For example, the single module may include one or more coils that can wirelessly communicate power as well as data/information. Optionally, the power module and the communication module may be integrated with the control circuit arrangement.

Optionally, the control circuit arrangement may be provided at least partly by a flexible circuit board and circuit elements operably coupled with (e.g., mounted on) the flexible circuit board. For example, the power module may be provided by at least part of flexible circuit board and/or at least one of the circuit elements. For example, the communication module may be provided by at least part of flexible circuit board and/or at least one of the circuit elements. For example, control circuit arrangement may be provided by at least part of flexible circuit board and/or at least one of the circuit elements.

Optionally, the one or more odor manipulation units comprises an odor manipulator. In some embodiments, each of the one or more odor manipulation units respectively comprises one or multiple ones of such odor manipulator. Different ones of the odor manipulators may provide or affect the same smell perceivable by the user, or different ones of the odor manipulators may provide or affect different smell perceivable by the user.

Optionally, the odor manipulator comprises: a body defining a chamber for receiving one or more chemical substances, and an interface through which the one or more chemical substances can be released from the body to provide or affect smell perceivable by the user. The chamber can be suitably shaped and/or sized. The interface may include one or more through-holes through which the one or more chemical substances can pass (e.g., when the one or more chemical substances is volatile) and/or one or more permeable parts through which the one or more chemical substances can permeate or penetrate (e.g., when the one or more chemical substances is volatile). The interface may be configured such that the one or more chemical substances received in the chamber is always in fluid communication with the one or more through-holes, e.g., without blocking means (valves or the like) for completely blocking the one or more through-holes. The one or more permeable parts may be provided by surface(s), window(s), etc.

Optionally, the odor manipulator further includes the one or more chemical substances received in the chamber. Optionally, the one or more chemical substances can provide or generate an odor to affect smell perceivable by the user. Hence, the odor manipulator may correspond to an odor generator. Optionally, the one or more chemical substances can react or interact with one or more substances in an environment the wearable electronic device is in to affect smell perceivable by the user. For example, the one or more chemical substances may affect (e.g., reduce, remove, provide, enhance, etc.) an odor present in the environment. The one or more chemical substances itself/themselves may be odorless or the one or more chemical substances itself/themselves may provide a smell. The smell or odor may be pleasant (e.g., fragrant, scent, aroma, etc.) or unpleasant (e.g., foul, etc.).

Optionally, the one or more chemical substances may be included in a phase change medium (phase change material(s)) arranged to be received in the chamber. When the phase change medium dissolves or vaporizes, the one or more chemical substances may be released, volatilized, or diffused from the body through the interface. When the phase change medium or materials condenses, the release, volatilization, or diffusion of one or more chemical substances from the body through the interface may be prevented, reduced, or stopped.

Optionally, the one or more chemical substances are included in a medium arranged to be received in the chamber. Optionally, the medium includes phase change material(s) that can be dissolved or vaporized to facilitate release of the one or more chemical substances from the body through the interface and/or can be condensed to prevent, reduce, or stop release of the one or more chemical substances from the body through the interface. Optionally, the odor manipulator further comprises the medium (with the one or more chemical substances) received in the chamber. The medium may include wax such as paraffin wax.

Optionally, the body may include a frame and a base coupled to the frame. The frame may be made of plastic material(s), such as polydimethylsiloxane (PDMS). The frame may be in the form of a ring, such as a generally rectangular (e.g., square) shaped ring, or the like. The base may be made of plastic material(s), such as polyethylene terephthalate (PET), polyimide (PI), etc. The base may be in the form of a film. The base may be generally planar or curved. The frame and the base may provide or define at least part of the chamber.

Optionally, the body may also include a shield providing the interface (e.g., the one or more through-holes of the interface). In some embodiments, the shield may be shaped and/or sized to prevent unwanted escape or removal of non-gaseous phase of the medium from the chamber. The shield may have a generally frusto-pyramidal or generally frusto-conical form, also with one or more through-holes.

Optionally, the odor manipulator further comprises: a release control mechanism arranged at least partly in the body and operable to control release of the one or more chemical substances from the body through the interface.

Optionally, the release control mechanism comprises a heating mechanism with one or more heating elements operable to provide heat to facilitate release of the one or more chemical substances from the body through the interface. The one or more heating elements may be in direct or indirect thermal contact with the one or more chemical substances, to provide heat to the one or more chemical substances.

Optionally, the one or more heating elements comprise one or more heating electrodes such as one or more Au/Cr electrodes, Au electrodes, Cu electrodes, etc. In some embodiments, the one or more heating electrodes may be arranged in or on a substrate, which may be made of plastic material(s) such as polyethylene terephthalate (PET), polyimide (PI), etc.

Optionally, the release control mechanism further comprises a temperature sensor for providing temperature information that indicates a temperature in the chamber or a temperature of the one or more heating elements. The one or more heating elements may be one or more heating electrodes.

Optionally, the one or more heating electrodes are operable as at least part of the temperature sensor.

Optionally, the release control mechanism further comprises a movement mechanism operable to move (e.g., one or more of: pivot, rotate, translate, etc.) the one or more heating elements relative to a frame of the body to facilitate cooling of the heating mechanism and hence reduce or stop vaporization of the one or more chemical substances. Optionally, the movement mechanism is further operable to move (e.g., one or more of: pivot, rotate, translate, etc.) the one or more chemical substances and/or the medium relative to the frame of the body. The movement mechanism may function as an active cooling mechanism.

Optionally, the movement mechanism comprises: a support member supporting at least the one or more heating elements; and an actuation mechanism coupled with the support member to move the support member and hence the one or more heating elements relative to the frame of the body. The support member may be made of plastic material(s), such as polyethylene terephthalate (PET), polyimide (PI), etc., and may be in the form of a film.

Optionally, the actuation mechanism may be a magnetic or electromagnetic actuation mechanism. In some embodiments, the actuation mechanism comprises a magnetic member and an electromagnetic member operable to interact to cause relative movement of the magnetic member and the electromagnetic member. In some embodiments, the relative movement includes movement of electromagnetic member (relative to the magnetic member). The relative movement of the magnetic member and the electromagnetic member is arranged to cause movement of the support member (and any components coupled to the support member). Movement of the support member may include, at least, cantilever motion and/or pivoting motion. The magnetic member may be a permanent magnet. The electromagnetic member may include an electromagnetic coil. In some embodiments, the magnetic or electromagnetic actuation mechanism may also help with temperature control.

Optionally, the release control mechanism further comprises: a temperature control circuit arrangement operably connected with the one or more heating elements and the temperature sensor for controlling operation (e.g., temperature) of the one or more heating elements based on temperature information provided by the temperature sensor. The temperature control circuit arrangement may include at least one processor, with one or more of: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), or any other digital or analog circuitry/circuitries configured to interpret and/or to execute program instructions and/or to process signals and/or information and/or data. In some embodiments, the temperature control circuit arrangement may be integrated with the control circuit arrangement of the wearable electronic device.

Optionally, the release control mechanism further comprises: a power control circuit arrangement operably connected with the electromagnetic member and the temperature sensor for controlling electric current in the electromagnetic member and hence movement (e.g., one or more of: movement timing, movement frequency, movement duration, movement amplitude, etc.) of the one or more heating elements relative to a frame of the body based on temperature information provided by the temperature sensor. The power control circuit arrangement may include at least one processor, with one or more of: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), or any other digital or analog circuitry/circuitries configured to interpret and/or to execute program instructions and/or to process signals and/or information and/or data. In some embodiments, the power control circuit arrangement may be integrated with the control circuit arrangement of the wearable electronic device.

In some embodiments in which the release control mechanism comprises both the power control circuit arrangement and the temperature control circuit arrangement, the power control circuit arrangement and the temperature control circuit arrangement may be separately arranged (e.g., provided by different circuits, processors, etc.). In some embodiments in which the release control mechanism comprises both the power control circuit arrangement and the temperature control circuit arrangement, the power control circuit arrangement and the temperature control circuit arrangement may be at least partly integrated (e.g., at least partly provided by the same circuit, the same processor, etc.).

Optionally, the wearable electronic device is configured to be skin-worn.

Optionally, the wearable electronic device is flexible and/or elastomeric.

Optionally, the wearable electronic device is a cordless electronic device.

Optionally, the wearable electronic device is configured for use by user with sensory impairment such as user with vision and/or hearing problem (e.g., user that is deaf and/or blind).

The user of the wearable electronic device may be human or animal.

In a second aspect, there is provided an odor manipulation unit of the wearable electronic device of the first aspect. One or more features of the odor manipulation unit in the first aspect may be applicable to the odor manipulation unit of the second aspect.

In a third aspect, there is provided an odor manipulator of an odor manipulation unit of the wearable electronic device of the first aspect. One or more features of the odor manipulator in the first aspect may be applicable to the odor manipulator of the third aspect.

In a fourth aspect, there is provided an odor manipulator for a wearable electronic device. The odor manipulator may be the odor manipulator of the first aspect. One or more features of the odor manipulator in the first aspect may be applicable to the odor manipulator of the fourth aspect (with or without reference to one or more parts of the wearable electronic device). The wearable electronic device may be the wearable electronic device of the first aspect.

Other features and aspects of the invention will become apparent by consideration of the detailed description and accompanying drawings. Any feature(s) described herein in relation to one aspect or embodiment may be combined with any other feature(s) described herein in relation to any other aspect or embodiment as appropriate and applicable.

Terms of degree such that “generally”, “about”, “substantially”, or the like, are used, depending on context, to account for manufacture tolerance, degradation, trend, tendency, imperfect practical condition(s), etc. For example, when a value is modified by a term of degree, such as “about”, such expression may include the stated value ±20%, ±10%, ±5%, ±2%, or ±1%.

As used herein, unless otherwise specified, the terms “connected”, “coupled”, “mounted”, etc., are intended to encompass both direct and indirect connection, coupling, mounting, etc. Also, unless otherwise specified, the terms “smell”, “odor”, and their equivalents, are intended to cover smell/odor that is/are pleasant or unpleasant (which can be subjective).

### BRIEF DESCRIPTION OF THE DRAWINGS

Embodiments of the invention will now be described, by way of example, with reference to the accompanying drawings in which:

FIG. 1 is a block diagram of a wearable electronic device in some embodiments of the invention;

FIG. 2 is a block diagram of an odor manipulator in some embodiments of the invention;

FIG. 3 is a block diagram of an odor manipulator in some embodiments of the invention;

FIG. 4 is a block diagram of an odor manipulator in some embodiments of the invention;

FIG. 5A is a schematic diagram (perspective view) of a wearable electronic device in one embodiment of the invention;

FIG. 5B is a schematic diagram (exploded view) of the wearable electronic device of FIG. 5A;

FIG. 6A is a schematic diagram (exploded view) of an odor manipulator in one embodiment of the invention;

FIG. 6B is a schematic diagram of the odor manipulator of FIG. 6A;

FIG. 7 is a photograph showing a portion of an odor manipulator in one embodiment of the invention;

FIG. 8 is a graph showing measured resistance of the heating electrodes of the odor manipulator of FIG. 7 at different temperatures of the heating electrodes;

FIG. 9 is a graph showing measured change in temperature in the odor manipulator of FIG. 7 with and without operation of the movement-mechanism-based cooling system when the heating temperature of the heating-electrodes is switched from 45° C. to 50° C. and then back to 45° C., from 45° C. to 55° C. and then back to 45° C., and from 45° C. to 60° C. and then back to 45° C., respectively;

FIG. 10 is a graph showing response time of the odor manipulator of FIG. 7 with and without operation of the movement-mechanism-based cooling system for the different temperature change statuses (Status A1: 45° C. to 50° C., Status A2: 45° C. to 55° C., Status A3: 45° C. to 60° C., Status B1: 50° C. to 45° C., Status B2: 55° C. to 45° C., Status B3: 60° C. to 45° C.);

FIG. 11 is a graph showing measured change in temperature in the odor manipulator of FIG. 7 for PET film (as support member) of different thicknesses (25 μm, 50 μm, 75 μm) when the heating temperature of the heating electrodes is switched from 45° C. to 50° C. and then back to 45° C., from 45° C. to 55° C. and then back to 45° C., and from 45° C. to 60° C. and then back to 45° C., respectively;

FIG. 12 is a graph showing response time of the odor manipulator of FIG. 7 with PET film (as support member) of different thicknesses (25 μm, 50 μm, 75 μm) for the different temperature change statuses (Status A1: 45° C. to 50° C., Status A2: 45° C. to 55° C., Status A3: 45° C. to 60° C., Status B1: 50° C. to 45° C., Status B2: 55° C. to 45° C., Status B3: 60° C. to 45° C.);

FIG. 13 is a graph showing temperature fluctuation in the odor manipulator of FIG. 7 for PET film (as support member) of different thicknesses (25 μm, 50 um, 75 μm) for different stabilized temperatures (45° C., 50° C., 55° C., 60° C.);

FIG. 14 is a graph showing measured change in temperature in the odor manipulator of FIG. 7 for different power input (0.16 W, 0.25 W, 0.3 W) to the heating electrodes when the heating temperature of the heating electrodes is switched from 45° C. to 50° C. and then back to 45° C., from 45° C. to 55° C. and then back to 45° C., and from 45° C. to 60° C. and then back to 45° C., respectively;

FIG. 15 is a graph showing response time of the odor manipulator of FIG. 7 for different power input (0.16 W, 0.25 W, 0.3 W) to the heating electrodes for the different temperature change statuses (Status A1: 45° C. to 50° C., Status A2: 45° C. to 55° C., Status A3: 45° C. to 60° C., Status B1: 50° C. to 45° C., Status B2: 55° C. to 45° C., Status B3: 60° C. to 45° C.);

FIG. 16 is a graph showing temperature fluctuation in the odor manipulator of FIG. 7 for different power input (0.16 W, 0.25 W, 0.3 W) to the heating electrodes for different stabilized temperatures (45° C., 50° C., 55° C., 60° C.);

FIG. 17 is a graph showing measured change in temperature in the odor manipulator of FIG. 7 for different power input (0.24 W, 0.3 W, 0.35 W) to the electromagnetic coil, when the heating temperature of the heating electrodes is switched from 45° C. to 60° C. and then back to 45° C.;

FIG. 18 is a graph showing response time of the odor manipulator of FIG. 7 for different power input (0.24 W, 0.3 W, 0.35 W) to the electromagnetic coil, when the heating temperature of the heating electrodes is switched from 45° C. to 60° C. and then from 60° C. back to 45° C.;

FIG. 19 is a graph showing measured temperature fluctuation in the odor manipulator of FIG. 7 for different power input (0.24 W, 0.3 W, 0.35 W) to the electromagnetic coil;

FIG. 20 shows a series of thermal images showing temperature response of the odor manipulator of FIG. 7 for different target temperatures (45° C., 50° C., 55° C., 60° C.); and

FIG. 21 is a graph showing duration of odor provided by the odor manipulator of FIG. 7 for eight different odor types (1—orange, 2—clove, 3—rosemary, 4—morning, 5—green tea, 6—clary sage, 7—mojito, 8—lavender), of different wax masses (0.0025 g, 0.01 g, 0.02 g) when the heating temperature of the heating electrodes is 60° C.

### DETAILED DESCRIPTION

The invention generally relates to a wearable electronic device for providing or affecting smell. The wearable electronic device may include one or more odor manipulation units each respectively operable to provide or affect smell perceivable by a user of the wearable electronic device, and a control circuit arrangement for operating the one or more odor manipulation units. Each of the one or more odor manipulation units may respectively include one or more odor manipulators. Odor manipulator that can generate an odor can be referred to as odor generator.

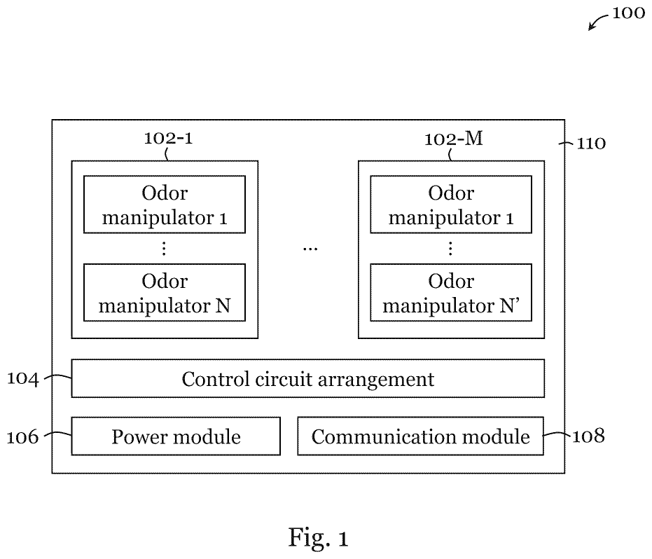

FIG. 1 illustrates a wearable electronic device 100 for providing or affecting smell in some embodiments of the invention. The wearable electronic device 100 includes multiple odor manipulation units 102-1, . . . , 102-M (M>1). Each of the odor manipulation units 102-1, . . . , 102-M is respectively operable to provide or affect smell perceivable by a user of the wearable electronic device 100. In some embodiments, some or all odor manipulation units 102-1, . . . , 102-M may be arranged to provide or affect different smell (e.g., different spatial and/or temporal odor profiles). In some embodiments, some or all odor manipulation units 102-1, . . . , 102-M may be arranged to provide or affect the same smell (e.g., the same spatial and/or temporal odor profile). As illustrated in FIG. 1, each of the odor manipulation units 102-1, . . . , 102-M respectively includes multiple odor manipulators (N>1, N′>1, N and N′ can be the same or different). In some embodiments, some or all odor manipulators of the same odor manipulation unit 102-1, . . . , 102-M may be arranged to provide or affect different smell (e.g., different spatial and/or temporal odor profiles). In some embodiments, some or all odor manipulators of the same odor manipulation unit 102-1, . . . , 102-M may be arranged to provide or affect the same smell (e.g., the same spatial and/or temporal odor profile). The odor manipulator may include chemical substance(s) that can provide or generate an odor to affect smell perceivable by the user, or chemical substance(s) that can react or interact with substance(s) in an environment the wearable electronic device is in to affect smell perceivable by the user.

The wearable electronic device 100 further includes a control circuit arrangement 104 operably coupled with the odor manipulation units 102-1, . . . , 102-M for operating the odor manipulation units 102-1, . . . , 102-M. The control circuit arrangement 104 may be formed by various circuit elements or modules, which may optionally be arranged on a circuit board (e.g., a flexible circuit board). In some embodiments, the control circuit arrangement 104 may independently operate some or all of the odor manipulation units 102-1, . . . , 102-M. In some embodiments, the control circuit arrangement 104 may simultaneously or selectively operate the some or all of the odor manipulation units 102-1, . . . , 102-M. In some embodiments, the control circuit arrangement 104 may independently operate some or all of the odor manipulators in the same odor manipulation unit 102-1, . . . , 102-M. In some embodiments, the control circuit arrangement 104 may simultaneously or selectively operate some or all of the odor manipulators in the same odor manipulation unit 102-1, . . . , 102-M. In some embodiments, the control circuit arrangement 104 may control timing, duration, and/or extent of activation and/or deactivation of each of the odor manipulators of the same odor manipulation unit to affect spatial and/or temporal odor profile (e.g., type(s), timing, intensity, and/or duration of odor) provided by the corresponding odor manipulation unit. In some examples, the control circuit arrangement 104 may include processor and memory storing one or more sets of control instructions each arranged to be operated by the processor to control respective (e.g., timing, duration, and/or extent of) activation and/or deactivation of one or more of the odor manipulators of one or more of the odor manipulation units 102-1, . . . , 102-M. Each of the set(s) of control instructions may be associated with a respective message recognizable by the user. For example, each of the one or more processors may include one or more: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), or any other digital or analog circuitry/circuitries configured to interpret and/or to execute program instructions and/or to process signals and/or information and/or data. For example, the memory may include: one or more volatile memory (such as RAM, DRAM, SRAM, etc.), one or more non-volatile memory (such as ROM, PROM, EPROM, EEPROM, FRAM, MRAM, FLASH, SSD, NAND, NVDIMM, etc.), or any of their combinations.

The wearable electronic device 100 further includes a power module 106 and a communication module 108. The power module 106 is operable to wirelessly receive power from an external device for operating the wearable electronic device 100. The power module 106 may be formed by various circuit elements or modules, such as inductive coil(s), energy storage element(s) (e.g., rechargeable battery/batteries, capacitor(s)), etc. These circuit elements or modules may optionally be arranged on a circuit board (e.g., a flexible circuit board). The communication module 108 is operable to wirelessly communicating data and/or instructions with (e.g., receiving data and/or instructions from) an external electronic device. The data and/or instructions may include data and/or instructions for controlling operation of the odor manipulation units 102-1, . . . , 102-M. The communication module may be formed by various circuit elements or modules and it may include: a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, above 5G, or the like) transceiver, an optical port, an infrared port, a USB connection interface, etc. These circuit elements or modules may optionally be arranged on a circuit board (e.g., a flexible circuit board). In one example, the power module 106 and the communication module 108 may be integrated by using coil(s) that can wirelessly communicate power as well as data/information. In one example, the power module 106 and the communication module 108 may be integrated with the control circuit arrangement 104 such that they have shared or common circuit elements or modules and may be at least partly arranged on the same circuit board (e.g., flexible circuit board).

The wearable electronic device 100 also includes a substrate 110. The odor manipulation units 102-1, . . . , 102-M, the control circuit arrangement 104, the power module 106, and the communication module 108 may be supported by or mounted to the substrate 110. The substrate 110 may be flexible and/or elastomeric. In some examples, the substrate 110 may be made of plastic material(s), such as polydimethylsiloxane (PDMS), soft silicon elastomer such as Ecoflex™, silicone rubber, etc. In some examples, the substrate 110 may be made of hydrogel. In some embodiments, the substrate 110 may include multiple substrate layers. At least part of the control circuit arrangement 104 may be arranged between different substrate layers. The substrate 110 may include cut-out(s) or opening(s) through which at least part of each odor manipulation units 102-1, . . . , 102-M could at least partly extend. In some embodiments, the substrate 110 is constructed (e.g., shaped and/or sized and/or made of suitable material(s)) to enable or facilitate wearing of the wearable electronic device 100 on a face of the user, e.g., on or near a nose of the user. In one example, the substrate 110 is constructed (e.g., shaped and/or sized and/or made of suitable material(s)) to enable or facilitate wearing at least part of the wearable electronic device 100 between a nose and a mouth (e.g., upper lip) of the user. In some embodiments, when the wearable electronic device 100 is worn on or near the nose of the user, at least one of the odor manipulation units 102-1, . . . , 102-M is configured (e.g., shaped and/or sized and/or supported by or mounted to the substrate in such a way) to be placed closer to one nostril of the user and at least another one of the odor manipulation units 102-1, . . . , 102-M is configured (e.g., shaped and/or sized and/or supported by or mounted to the substrate in such a way) to be placed closer to another nostril of the user.

In some embodiments, the wearable electronic device 100 is configured to be skin-worn, with suitable means for adhering directly to the skin of the user. In some embodiments, the wearable electronic device 100 is, like the substrate 110, flexible and/or elastomeric, even if some parts of the odor manipulators may be relatively rigid. In some embodiments, the wearable electronic device 100 is configured for use by user with sensory impairment such as user with vision and/or hearing problem (e.g., user that is deaf and/or blind), to provide to the user respective odor-based message recognizable by the user (who may have been trained to associate different odor profiles with different messages). The user may be human or animal.

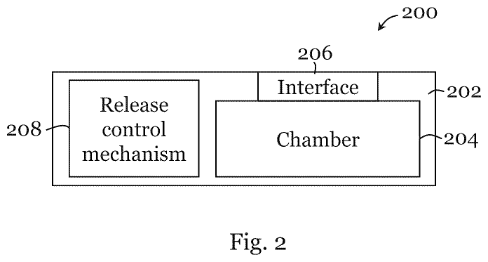

FIG. 2 illustrates an odor manipulator 200 in some embodiments of the invention. The odor manipulator 200 may be used as the odor manipulator in any of the odor manipulation units 102-1, . . . , 102-M of FIG. 1. The odor manipulator 200 generally includes a body 202 defining: a chamber 204 for receiving chemical substance(s), and an interface 206 through which the chemical substance(s) can be released from the body 202 to provide or affect smell perceivable by the user. The odor manipulator 200 further includes a release control mechanism 208 arranged at least partly in the body 202 and operable to control release of the chemical substance(s) from the body 202 through the interface 206. The body 202 may be made of plastic material(s) and may include different body parts or portions.

The chamber 204 can be shaped and/or sized according to applications. In some embodiments, the interface 206 includes through-hole(s) through which the chemical substance(s), when volatile, can pass. The interface 206 may be configured such that the chemical substance(s) received in the chamber 204 is always in fluid communication with the through-hole(s). In some embodiments, the interface 206 includes permeable part(s) through which the chemical substance(s), when volatile, can permeate or penetrate. The permeable part(s) may be provided by surface(s), window(s), etc. The chemical substance(s) receivable or received in the chamber 204 can (i) provide or generate an odor to affect smell perceivable by the user or (ii) react or interact with substance(s) in an environment to affect smell perceivable by the user (e.g., affect (e.g., reduce, remove, provide, enhance, etc.) an odor present in the environment). The chemical substance(s) may be odorless or odorous (e.g., pleasant or unpleasant). In some embodiments, the chemical substance(s) may be included in a phase change medium (phase change material(s)) arranged to be received in the chamber 204. When the phase change medium dissolves or vaporizes, the chemical substance(s) may be released, volatilized, or diffused from the body 202 through the interface 206. When the phase change medium or materials condenses, the release, volatilization, or diffusion of one or more chemical substances from the body 202 through the interface 206 may be prevented, reduce, or stopped. In some embodiments, the phase change medium may include wax such as paraffin wax.

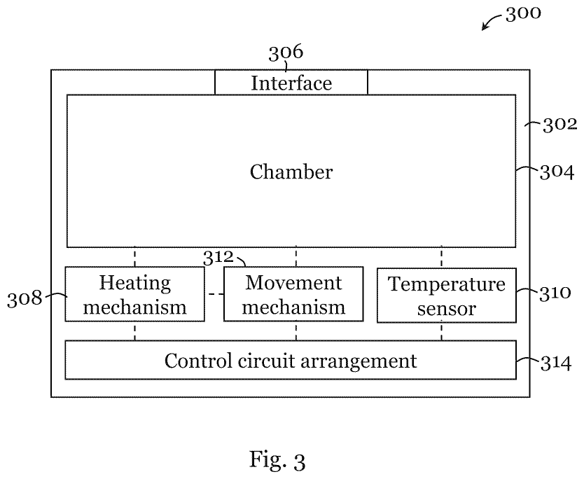

FIG. 3 illustrates an odor manipulator 300 in some embodiments of the invention. The odor manipulator 300 can be considered as a specific example of the odor manipulator 200 of FIG. 2. Like the odor manipulator 200, the odor manipulator 300 includes a body 302 defining a chamber 304 and an interface 306. As the body 302, chamber 304, and interface 306 can be generally the same as the body 202, chamber 204, and interface 206, related details are not repeated here. Features of the chemical substance(s) and/or the medium provided with reference to the odor manipulator 200 are also applicable to the odor manipulator 300 hence related details are not repeated here. Like the odor manipulator 200, the odor manipulator 300 includes a release control mechanism arranged at least partly in the body 302 and operable to control release of the chemical substance(s) from the body 302 through the interface 306. The release control mechanism includes a heating mechanism 308, a temperature sensor 310, a movement mechanism 312, and a control circuit arrangement 314.

The heating mechanism 308 may include one or more heating elements operable to provide heat directly or indirectly to the chemical substance(s), to facilitate release of the chemical substance(s) from the body 302 through the interface 306. The one or more heating elements may include electrode(s), such as Au/Cr electrode(s), Au electrodes, Cu electrodes, etc., which may be optionally arranged on or in a substrate, made, e.g., of plastic material(s) such as polyimide (PI).

The temperature sensor 310 may be arranged to provide temperature information that indicates: a temperature in the chamber 304 or a temperature of the heating mechanism 308 (such as the one or more heating elements). The temperature sensor 310 may include electrode, thermistor, thermocouple, resistance temperature detector (RTD), etc., arranged directly in the chamber 304 or otherwise thermally coupled with the chamber 304.

In some embodiments, the temperature sensor 310 may be integrated with the heating mechanism 308. For example, the integrated temperature sensor 310 and heating mechanism 308 may be provided by the heating element(s), e.g., the heating electrode(s) such as Au/Cr, Au, or Cu electrode(s).

The movement mechanism 312 may function as an active cooling mechanism. The movement mechanism 312 may be operable to move at least part of the heating mechanism 308, such as the heating element(s), relative to a frame of the body 302 to facilitate cooling of the heating mechanism and hence reduce or stop vaporization of the chemical substance(s). For example, the movement mechanism 312 may be operable to pivot, rotate, and/or translate at least part of the heating mechanism 308, such as the heating element(s), relative to the frame of the body 302. In some embodiments, the movement mechanism 312 may also move the chemical substance(s) and/or the medium relative to the frame of the body 302. For example, the movement mechanism 312 may be operable to pivot, rotate, and/or translate the chemical substance(s) and/or the medium relative to the frame of the body 302.

The control circuit arrangement 314 may be arranged to control operation of the heating mechanism 308 and/or the movement mechanism based at least in part on the temperature information provided by the temperature sensor 310. In some embodiments, the control circuit arrangement 314 may be arranged to control operation (hence temperature) of at least part of the heating mechanism 308 (such as the one or more heating elements) based on temperature information provided by the temperature sensor 310. For example, the control circuit arrangement 314 may monitor the temperature of the one or more heating elements or in the chamber 304 using the temperature sensor 310 to determine whether the temperature of the heating mechanism 308 or the chamber 304 has reached a target temperature or is within a temperature range. In some embodiments, the control circuit arrangement 314 may be arranged to control operation of the controlling electric current to the movement mechanism 312 and hence relative movement of at least part of the heating mechanism 308 (such as the one or more heating elements) relative to the frame of the body 302 based on temperature information provided by the temperature sensor 310. The control circuit arrangement 314 may include multiple circuit parts or modules each serving a respective function, or the control circuit arrangement 314 may include one or more parts that provide multiple functions. In some embodiments in which the odor manipulator 300 is used in the wearable electronic device 100, the control circuit arrangement 314 may be at least partly integrated with the control circuit arrangement 104 (e.g., arranged in the same circuit arrangement). In some embodiments, the control circuit arrangement 314 may be implemented using at least one processor, with one or more of: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), or any other digital or analog circuitry/circuitries configured to interpret and/or to execute program instructions and/or to process signals and/or information and/or data. Although in FIG. 3 the control circuit arrangement 314 is shown to be included in the body 302, in some embodiments the control circuit arrangement 314 may be arranged at least partly external to the body 302.

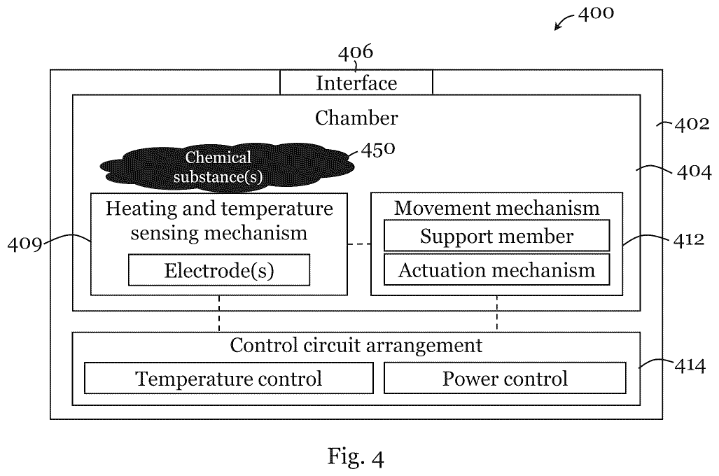

FIG. 4 illustrates an odor manipulator 400 in some embodiments of the invention. The odor manipulator 400 can be considered as a specific example of the odor manipulator 200 of FIG. 2 or a specific example of the odor manipulator 300 of FIG. 3. Like the odor manipulators 200, 300, the odor manipulator 400 includes a body 402 defining a chamber 404 and an interface 406. As the body 402, chamber 404, and interface 406 can be generally the same as the body 202, 302, chamber 204, 304, and interface 206, 306, related details are not repeated here. FIG. 4 also shows the chemical substance(s) 450 received in the chamber 404. The chemical substance(s) 450 may be included (e.g., embedded) in a phase change material medium. Features of the chemical substance(s) 450 and/or the medium provided with reference to the odor manipulators 200, 300 are also applicable to the odor manipulator 400 hence related details are not repeated here. Like the odor manipulators 200, 300, the odor manipulator 400 includes a release control mechanism arranged at least partly in the body 402 and operable to control release of the chemical substance(s) 450 from the body 402 through the interface 406. Like the odor manipulator 300, the release control mechanism includes a heating and temperature sensing mechanism 409 (integrated heating mechanism and temperature sensor), a movement mechanism 412, and a control circuit arrangement 414. The following description focuses on the technical details or differences not specifically presented above with reference to the odor manipulator 300 of FIG. 3.

In the embodiments of FIG. 4, the heating and temperature sensing mechanism 409 may generally correspond to (e.g., serve the same general function as) the heating mechanism 308 and the temperature sensor 310 integrated. The heating and temperature sensing mechanism 409 may provide heat directly or indirectly to the chemical substance(s) 450 to facilitate release of the chemical substance(s) 450 from the body 402 through the interface 406 and may provide temperature information that indicates: a temperature in the chamber 404 or a temperature of at least part of the heating and temperature sensing mechanism 409 (itself). The heating and temperature sensing mechanism 409 includes electrode(s) such as Au/Cr electrode(s). The electrodes may include an Au layer and a Cr layer, which are optionally arranged on or in a substrate, which may be made, e.g., of plastic material(s) such as polyimide (PI). The electrode(s) may be heating electrode(s) operable to provide heat to/in the body 402, and the electrical properties (e.g., resistance) of the heating electrode(s) may provide the temperature information.

In the embodiments of FIG. 4, the movement mechanism 412 may generally correspond to (e.g., serve the same general function as) the movement mechanism 312. The movement mechanism 412 may function as an active cooling mechanism, to move at least part of the heating and temperature sensing mechanism 409, such as the heating electrode(s), relative to a frame of the body 402 to facilitate cooling of the heating and temperature sensing mechanism 409, such as the heating electrode(s), and hence reduce or stop vaporization of the chemical substance(s) 450. The movement mechanism 412 may further move the chemical substance(s) 450 and/or the medium relative to the frame of the body 402 to further facilitate cooling of the chemical substance(s) 450 hence further reduce or stop vaporization of the chemical substance(s) 450. In some embodiments, to this end, the movement mechanism 412 may enable or cause direct or indirect thermal contact between the heating electrode(s) (relatively hot) and one or more other parts (relatively cold) of the odor manipulator 400 (e.g., one or more parts of the movement mechanism 412), to facilitate heat dissipation away from the heating electrode(s).

The movement mechanism 412 includes a support member supporting the electrode(s) of the heating and temperature sensing mechanism 409 and an actuation mechanism coupled with the support member to move the support member and hence the electrode(s) of the heating and temperature sensing mechanism 409 relative to a frame of the body 402. The support member may be made of plastic material(s), such as polyethylene terephthalate (PET), and may be in the form of a film, which can be moved by the actuation mechanism relative to the frame of the body 402. In some embodiments, the actuation mechanism may be a magnetic or electromagnetic actuation mechanism, with a magnetic member and an electromagnetic member operable to interact to cause relative movement of the magnetic member and the electromagnetic member. The magnetic member may be a permanent magnet. The electromagnetic member may include an electromagnetic coil, e.g., made of copper. The support member may be coupled to either the magnetic member or the electromagnetic member, and the relative movement of the magnetic member and the electromagnetic member causes movement of the support member and component(s) coupled to it (e.g., the electrode(s) of the heating and temperature sensing mechanism 409) relative to the frame of the body 402. Movement of the support member and component(s) coupled to it may include, at least, cantilever motion and/or pivoting motion. The movement may enable or cause thermal contact between one or more parts of the odor manipulator 400 and the support member (and component(s) coupled to it) to facilitate cooling of the support member (and component(s) coupled to it).

In the embodiments of FIG. 4, the control circuit arrangement 414 may generally correspond to (e.g., serve the same general function as) the control circuit arrangement 314. The control circuit arrangement 414 may include a temperature control circuit operably coupled with the heating and temperature sensing mechanism 409 to control operation of the electrode(s) of the heating and temperature sensing mechanism 409 based at least in part on the temperature information it provides. The temperature control circuit can be used to control the temperature in the chamber 404 hence to control vaporization of the chemical substance(s). The control circuit arrangement 414 may further include a power control circuit operably coupled with the electromagnetic member of the actuation mechanism of the movement mechanism 412, as well as the heating and temperature sensing mechanism 409, to control electric current in the electromagnetic member and hence movement (e.g., one or more of: timing, frequency, duration, amplitude, etc.) of the electrode(s) of the heating and temperature sensing mechanism 409 relative to the frame of the body 402 based on temperature information provided by the electrode(s) of the heating and temperature sensing mechanism 409. The control circuit arrangement 414 may be implemented using at least one processor, with one or more of: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), or any other digital or analog circuitry/circuitries configured to interpret and/or to execute program instructions and/or to process signals and/or information and/or data. Although in FIG. 4, the control circuit arrangement 414 is shown to be included in the body 402, in some embodiments the control circuit arrangement 414 may be arranged at least partly external to the body 402. The power control circuit and the temperature control circuit may be separately arranged (e.g., provided by different circuits, processors, etc.), or at least partly integrated.

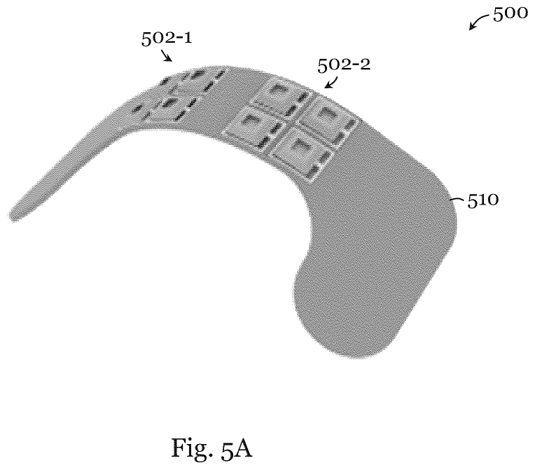

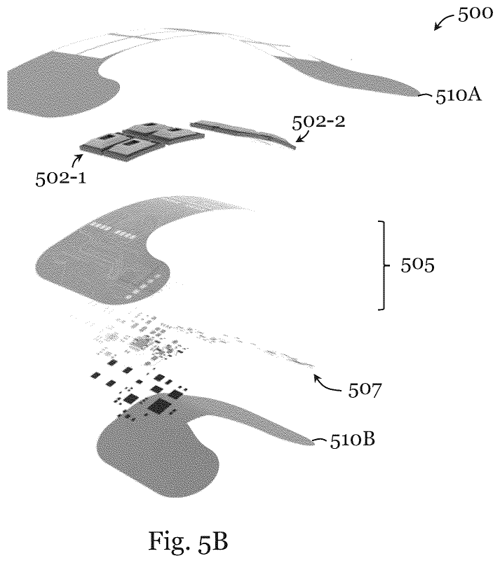

FIGS. 5A and 5B illustrate a wearable electronic device 500 for providing or affecting smell in one embodiment of the invention. The wearable electronic device 500 can be considered as a specific example of the wearable electronic device 100 of FIG. 1. The wearable electronic device 500 includes a substrate 510 and two odor manipulator units 502-1, 502-2 supported by the substrate 510. The odor manipulator units 502-1, 502-2 may be electro-thermal based odor manipulator units. In this embodiment, each of the odor manipulator units 502-1, 502-2 respective include four odor manipulators, such as but not limited to the ones described with reference to FIGS. 2 to 4. The four odor manipulators are arranged in a 2D array, with two rows and two columns. Each of the odor manipulator units 502-1, 502-2 is respectively arranged to provide or affect smell perceivable by a user of the wearable electronic device 500. In some embodiments, the odor manipulator units 502-1, 502-2 may provide the same type or types of smell. In some embodiments, different odor manipulators of the same unit odor manipulator units 502-1, 502-2 may provide or affect different smell.

As shown in FIG. 5B, the substrate 510 includes two substrate layers 510A, 510B. In this embodiment, the two substrate layers 510A, 510B are elastomeric polydimethylsiloxane (PDMS) layers. The substrate 510 has a generally U-shape profile, corresponding to the shape of the region around the upper lip of a user. The substrate 510 is arranged to be worn on the face of the user, near the nose, e.g., around at least part of the upper lip and at least partly between the nose and the upper lip. The odor manipulator units 502-1, 502-2 are disposed on the substrate 510 and are shaped and/or sized such that when the substrate 510 or the wearable electronic device 500 is worn around at least part of the upper lip one of the odor manipulator units 502-1, 502-2 is disposed closer to one nostril of the user e.g., between the nostril and the upper lip) and another one of the odor manipulator units 502-1, 502-2 is disposed closer to another one nostril of the user (e.g., between the other nostril and the upper lip), such that each of the odor manipulator units 502-1, 502-2 is mainly arranged to provide smell to a corresponding nostril.

In this embodiment, the first substrate layer 510A includes eight openings, which generally correspond to the eight odor manipulators of the odor manipulator units 502-1, 502-2 and through which respective odor manipulator of the odor manipulator units 502-1, 502-2 may extend. The wearable electronic device 500 further includes a flexible printed circuit board 505 with surface-mounted electrical elements 507, disposed between the two substrate layers 510A, 510B. The two substrate layers 510A, 510B help to prevent damage of the flexible printed circuit board 505 and the surface-mounted electrical elements 507. The substrate layer 510B is arranged for interfacing with the skin of the user so as to be skin-worn.

The flexible printed circuit board 505 with surface-mounted electrical elements 507 may provide the control circuit arrangement 104, the power module 106, and the communication module 108, as described with reference to FIG. 1. For brevity, details of these arrangements and modules are not repeated here. The control circuit arrangement provided by the flexible printed circuit board 505 with surface-mounted electrical elements 507 can control operation of the odor manipulator units 502-1, 502-2 in particular their odor manipulators. The control circuit arrangement provided by the flexible printed circuit board 505 with surface-mounted electrical elements 507 can independently control respective (e.g., timing, duration, and/or extent of) activation and/or deactivation of the odor manipulator units 502-1, 502-2 and/or their odor manipulators. As such, the odor manipulator units 502-1, 502-2 and/or their odor manipulators may be operated selectively or simultaneously. In this embodiment, the control circuit arrangement provided by the flexible printed circuit board 505 with surface-mounted electrical elements 507 include processor(s) and memory storing one or more sets of control instructions each arranged to be operated by the processor(s) to control respective (e.g., timing, duration, and/or extent of) activation and/or deactivation of the odor manipulator units 502-1, 502-2 and/or their odor manipulators. Each of the set(s) of control instructions may be associated with a respective message recognizable by a user who has been trained to associate different smell profiles with different messages. The flexible printed circuit board 505 with surface-mounted electrical elements 507 may include at least one coil arranged to operate as a power, data, and/or information transfer coil.

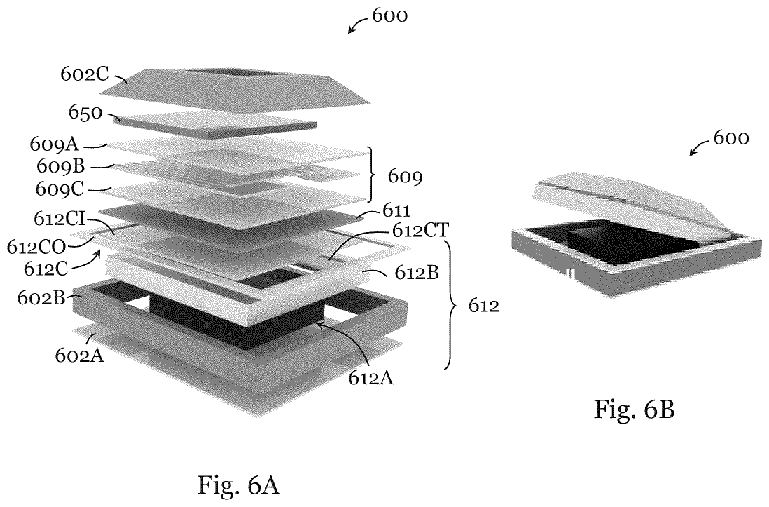

FIGS. 6A and 6B illustrate an odor manipulator 600 in one embodiment of the invention. The odor manipulator 600 can be considered as a specific example of the odor manipulator 400 of FIG. 4. The odor manipulator 600 can be used as one or more of the odor manipulators of the odor manipulator units 502-1, 502-2 of the wearable electronic device 500 in FIGS. 5A and 5B.

As shown in FIG. 6A, the odor manipulator 600 includes a body defining a chamber for receiving chemical substance(s) and an interface through which the chemical substance(s)-can be released from the body to provide or affect smell perceivable by the user. In this embodiment, the body includes a frame 602B in the form of a ring and a base 602A in the form of a generally planar film coupled (attached, bonded, integrated, etc.) to the frame 602B to close one end of the frame 602B. The frame 602B is made of polydimethylsiloxane (PDMS). The base is made of polyethylene terephthalate (PET). The frame 602B and the base 602A may provide or define at least part of the chamber. The chamber is used to house or receive, at least, the movement mechanism, which can be used to facilitate active cooling. In this embodiment, the body also includes a shield 602C in a generally inverted frusto-pyramidal form. The shield 602C may be coupled to the frame 602B and the base 602A. The shield 602C include a through-hole that act as the interface through which the chemical substance(s) can be released from the body to provide or affect smell perceivable by the user. In this embodiment, the shield 602C also helps to define the chamber. In this embodiment, a medium 650 including the chemical substance(s) is contained in the chamber. The medium 650 is an odorous wax, or paraffin wax, which is a phase change material that can be dissolved when sufficiently heated. In this embodiment, the medium 650 is in the form of a film or a small block. The shield 602C is shaped and sized to prevent unwanted escape or removal of (e.g., spilling of) non-gaseous phase of the medium 650 from the chamber.

The odor manipulator 600 also includes a release control mechanism, which includes a heating and temperature sensing mechanism 609 and a movement mechanism 612. In some embodiments, the heating and temperature sensing mechanism 609 and the movement mechanism 612 may function similarly or the same as the heating and temperature sensing mechanism 409 and a movement mechanism 412 of FIG. 4 so for brevity related details are not substantially repeated here.

The heating and temperature sensing mechanism 609 is operable to provide heat to the medium 650 to facilitate release of the chemical substance(s) from the body through the interface and to provide temperature information that indicates a temperature in the chamber or a temperature of one or more parts of the odor manipulator 600. In this embodiment, the heating and temperature sensing mechanism 609 comprises electrodes, which include Au/Cr electrodes. The Au/Cr electrodes may include Au/Cr materials 609B arranged on or in polyimide (PI) layers 609A, 609C. In this embodiment, the shield 602C is coupled to the PI layer 609A, to define a shape receiving the medium 650.

The movement mechanism 612 is operable to move electrodes of the heating and temperature sensing mechanism 609 relative to the frame of the body to facilitate cooling of the heating mechanism and hence reduce or stop vaporization of the chemical substance(s) included in the medium 650. In this embodiment, the movement mechanism 612 includes a disc-like permanent magnet 612A received in the space defined by the frame 602B (e.g., coupled to the base 602A), an electromagnetic coil 612B made of copper and arranged at least part around the permanent magnet 612A, and a polyethylene terephthalate (PET) film or layer 612C arranged on the electromagnetic coil 612B and operable to be in contact with the magnet 612A. In this embodiment, the magnet 612A and the frame 602B are both mounted on or fixed to the base 602A. The PET film 612C includes an outer periphery portion 612CO attached to the upper side of the frame 602B, an inner patch portion 612CI, and a bridge or tongue portion 612CT connecting the outer periphery portion 612CO and the inner patch portion 612CI. In this embodiment, the inner patch portion 612CI of the PET film 612C operates as a support member. The inner patch portion 612CI of the PET film 612C is attached, on one side, to the heating and temperature sensing mechanism 609, in particular the polyimide (PI) layer 609C, via an adhesive layer 611. The inner patch portion 612CI of the PET film 612C is attached, on another side, to the electromagnetic coil 612B. The electromagnetic coil 612B can be energized to magnetically interact with the permanent magnet 612A to cause relative movement between the permanent magnet 612A and the electromagnetic coil 612B and hence to move the inner patch portion 612CI of the PET film 612C (and the heating and temperature sensing mechanism 609 the medium 650, and the shield 602C) relative to the frame 602B and the base 602A. In this embodiment, the electromagnetic coil 612B and the inner patch portion 612CI of the PET film 612C, hence the adhesive layer 611, the heating and temperature sensing mechanism 609, the medium 650, and the shield 602C that are coupled to the inner patch portion 612CI of the PET film 612C, are movable (e.g., raised and lowered, in cantilever motion) relative to the permanent magnet 612A, when the electromagnetic coil 612B is energized. In other words, energization of the electromagnetic coil 612B at least moves the electromagnetic coil 612B and components coupled with it. Such movement can help with dissipating heat in the chamber, or the component(s) inside the chamber, to reduce or stop vaporization of the chemical substance(s) included in the medium 650. Such movement can also cause or enable thermal contact between the electrodes of the heating and temperature sensing mechanism 609 with one or more parts of the movement mechanism 612, which may help with heat dissipation.

Although not specifically illustrated, the release control mechanism also includes related temperature and power control circuit(s) for operating the heating and temperature sensing mechanism 609 and the movement mechanism 612. In some embodiments, the related control circuit(s) may function similarly or the same as the control circuit arrangement 414 of FIG. 4 so for brevity related details are not substantially repeated here. Generally, in this embodiment, the control circuit can control electric current in (e.g., provided to) the electromagnetic coil 612B to control the timing, frequency, duration, amplitude, etc. of the movement of the electromagnetic coil 612B hence the PET film 612C (in turn the heating and temperature sensing mechanism 609 attached to the PET film 612C, the medium 650 supported by the heating and temperature sensing mechanism 609, and the shield 602C coupled with the heating and temperature sensing mechanism 609), relative to the permanent magnet 612A or the frame 602B. Movement of the PET film 612C (and the heating and temperature sensing mechanism 609, the medium 650, and the shield 602C) may include, at least, cantilever motion or pivoting motion, as illustrated in FIG. 6B, enabled by movement of the inner patch portion of the PET film 612C relative to the outer periphery portion of the PET film 612C (which is fixedly attached to the frame 602B) via the bridge or tongue portion. Such movement may enable or create thermal contact that cools the PET film 612C and the attached heating and temperature sensing mechanism 609, the movement mechanism 612, and/or the medium 650.

In this embodiment, in operation, when power is provided to the electromagnetic coil 612B, the electromagnetic coil 612B and the permanent magnet 612A electromagnetically interact to cause cantilever movement of the electromagnetic coil 612B (and components coupled to it) relative to the permanent magnet 612A (and components coupled to it) such that the electromagnetic coil 612B selectively moves away from and towards the permanent magnet 612A. When the electromagnetic coil 612B (and components coupled to it) moves away from the permanent magnet 612A, the inner patch portion 612CI of the PET film 612C does not contact the permanent magnet 612A and heat generated by the energized electromagnetic coil 612B can facilitate heating of the Au/Cr electrodes (and components coupled to it). This is because compared with the case the electromagnetic coil 612B is not energized, the energized electromagnetic coil 612B is hotter and hence will less readily draw heat away from the heated Au/Cr electrodes (the heated Au/Cr electrodes are hotter than the electromagnetic coil 612B). When the electromagnetic coil 612B (and components coupled to it) moves towards the permanent magnet 612A, the inner patch portion 612CI of the PET film 612C contacts the permanent magnet 612A and heat generated by the Au/Cr electrodes (hotter) can be dissipated through the permanent magnet 612A (colder), via the inner patch portion 612CI of the PET film 612C, to facilitate cooling of the Au/Cr electrodes (and components coupled to it).

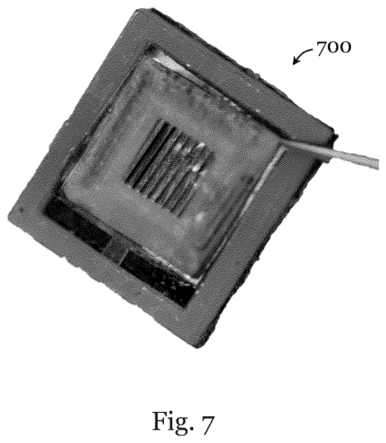

FIG. 7 shows a portion of an odor manipulator 700 made in accordance with the design of the odor manipulator 600 in one embodiment of the invention. The odor manipulator 700 in this embodiment is referred to as an odor generator (OG), as the chemical substance(s) it contains are odorous. FIG. 7 shows the frame receiving the heating electrodes, and a phase change medium including the chemical substance(s) placed on electrodes.

The odor generated by the odor manipulator 700 may be influenced by various factors such as temperature inside the chamber, the operation of the cooling system (movement mechanism), the PET film (support member) thickness, and energy input to the heating electrodes, etc. To investigate the electrical performance of the odor manipulator 700, experiments are conducted to verify the relationship between the electrical characteristic of the odor manipulator 700 and the various factors.

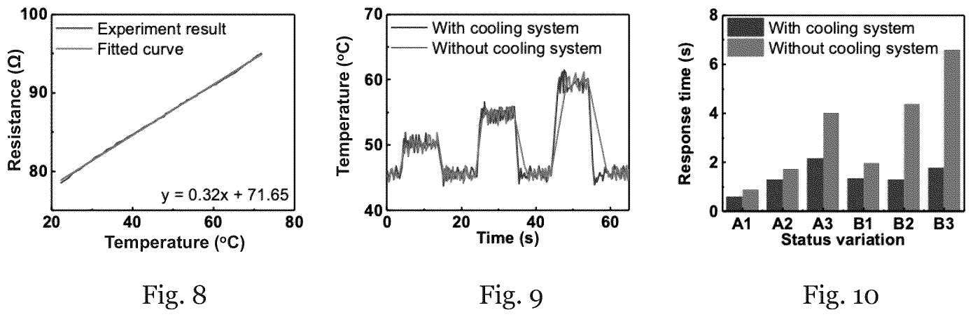

FIG. 8 shows the measured resistance of the heating electrodes of the odor manipulator 700 at different temperatures. The results show good linearity between the measured resistance of the electrodes and temperature ranging from 20° C. to 80° C.

Experiments are performed to investigate the effect of movement-mechanism-based cooling system on temperature change in the odor manipulator 700. In this example, the experimental conditions are as follows: power into heating electrodes=0.3 W, power into electromagnetic coil=0.24 W, environmental temperature=19° C., wax mass=0.0025 g, PET thickness=25 μm.

FIGS. 9 and 10 show the effect of movement-mechanism-based cooling system on temperature change in the odor manipulator 700. Specifically: FIG. 9 shows the measured change in temperature in the odor manipulator 700 with and without operation of the movement-mechanism-based cooling system when the heating temperature of the electrodes is switched from 45° C. to 50° C. and then back to 45° C., from 45° C. to 55° C. and then back to 45° C., and from 45° C. to 60° C. and then back to 45° C., respectively. FIG. 10 shows the response time of the odor manipulator 700 with and without operation of the movement-mechanism-based cooling system for different temperature change statuses (Status A1: 45° C. to 50° C., Status A2: 45° C. to 55° C., Status A3: 45° C. to 60° C., Status B1: 50° C. to 45° C., Status B2: 55° C. to 45° C., Status B3: 60° C. to 45° C.). From FIGS. 9 and 10, it can be seen that the use of the movement-mechanism-based cooling system can enable active cooling to more rapidly reduce the temperature of the electrodes and the wax. Operation of the movement-mechanism-based cooling system can lead to more rapid response time during cooling (Status B1: 50° C. to 45° C., Status B2: 55° C. to 45° C., Status B3: 60° C. to 45° C.). This may be due to thermal diffusion from the electrodes and the wax to the magnet. For the different cooling phases B1 to B3, the response times are similar. Operation of the movement-mechanism-based cooling system may also accelerate the heating process (Status A1: 45° C. to 50° C., Status A2: 45° C. to 55° C., Status A3: 45° C. to 60° C.) due to the heat produced by the electromagnetic system (e.g., heat generated by the energized electromagnetic coil) in thermal contact with the electrodes. The heat produced by the electromagnetic system reduces the thermal gradient between the electromagnetic system and the electrodes and reduce the heat dissipation from electrodes to electromagnetic system. This effect appears to be particularly prominent for Status A3 that has a relatively large heating range (from 45° C. to 60° C.), in which case the heating time is significantly reduced compared to without operation of the movement-mechanism-based cooling system.

Experiments are performed to investigate the effect of the thickness of the PET film (support member), disposed between the electromagnetic coils and the heating and temperature sensing electrodes, on the electrical response of the odor manipulator 700. Specifically, experiments are performed to study the temperature change, response time of temperature variation, and temperature fluctuation for different PET film thicknesses (25 μm, 50 μm, and 75 μm). In this example, the experimental conditions are as follows: power into heating electrodes=0.3 W, power into electromagnetic coil=0.24 W, environmental temperature=19° C., wax mass=0.0025 g.

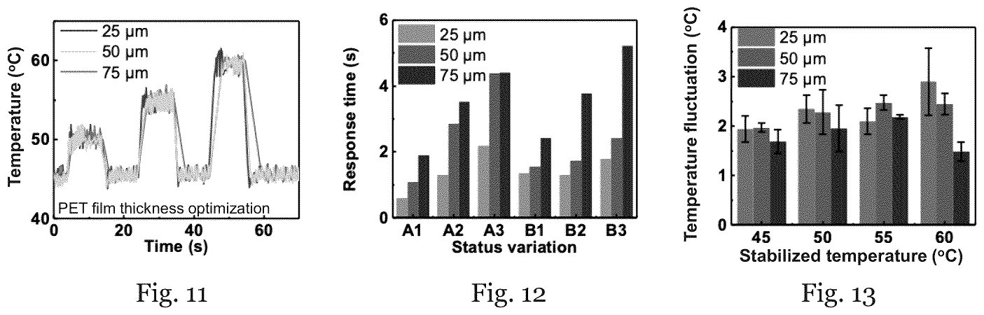

FIG. 11 shows the measured change in temperature in the odor manipulator 700 for PET film (as support member) of different thicknesses (25 μm, 50 μm, 75 μm) when the heating temperature of the electrodes is switched from 45° C. to 50° C. and then back to 45° C., from 45° C. to 55° C. and then back to 45° C., and from 45° C. to 60° C. and then back to 45° C., respectively. FIG. 12 shows the response time of the odor manipulator 700 with PET film (as support member) of different thicknesses (25 μm, 50 μm, 75 μm) for different temperature change statuses (Status A1: 45° C. to 50° C., Status A2: 45° C. to 55° C., Status A3: 45° C. to 60° C., Status B1: 50° C. to 45° C., Status B2: 55° C. to 45° C., Status B3: 60° C. to 45° C.). FIG. 13 shows temperature fluctuation in the odor manipulator 700 (of the heating electrodes) for PET film (as support member) of different thicknesses (25 μm, 50 μm, 75 μm) for different stabilized temperatures (45° C., 50° C., 55° C., 60° C.).

As shown in FIGS. 11 and 12, as the thickness of the PET film increases, the response time is slower during both heating and cooling phases (statuses A1 to A3 and B1 to B3). This is due to the fact that thicker material(s) generally require longer time for heat transfer. Thus, generally, the thinner the PET film, the better its heat transfer efficiency. During cooling, a thinner PET film has better thermal conductivity, and hence heat can more readily be transferred to the magnet that contacts the PET film, leading to faster cooling. As shown in FIG. 13, at certain temperatures from 45° C. to 60° C., the temperature fluctuation of the electrodes decreases as the thickness of the PET film increases. A relatively large temperature fluctuation can be observed for the 25 μm PET film because the 25 μm PET film is relatively thin, resulting in a relatively large perturbation of the corresponding resistance value and causing relatively large temperature fluctuation. It is noted that the thickness of the PET film would affect the movement, in particular the degree of lifting, of the PET film. Generally, a thicker PET film would have more resistance to bending tension and is less likely to move (e.g., lift) under extended load whereas a thinner PET film would have less resistance to bending tension and is easier to move (e.g., lift). Hence, a thinner PET film can more readily move (e.g., lift or bounce up) by the force generated by the interaction between the electromagnetic coil and the magnet during active cooling down. Therefore, a thinner PET film may provide faster cooling effect and larger movement capacity. In one example, a 25 μm PET film is used in the design of the odor manipulator 700.

Experiments are performed to investigate the effect of the power input to the electrodes on the corresponding electrical response of the heating temperature, the response time of temperature change, and the temperature fluctuation. In this experiment, different power input is applied to the electrodes to optimize the power input (0.16 W, 0.25 W, 0.3 W). In this example, the experimental conditions are as follows: power into electromagnetic coil=0.24 W, environmental temperature=19° C., wax mass=0.0025 g, and PET thickness=25 μm.

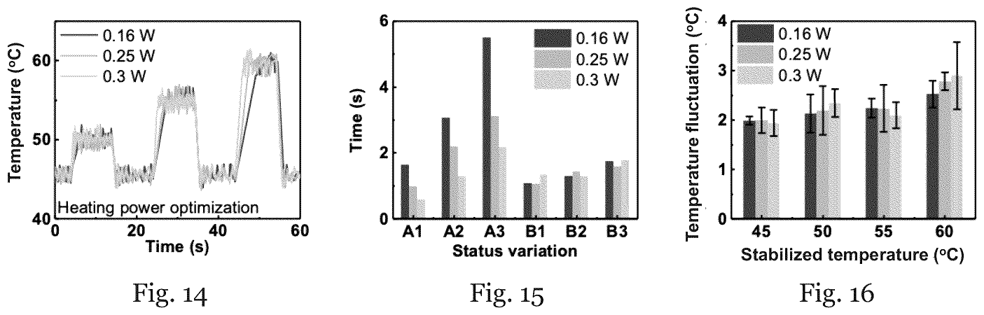

FIG. 14 shows the measured change in temperature in the odor manipulator 700 for different power input (0.16 W, 0.25 W, 0.3 W) to the heating electrodes when the heating temperature is switched from 45° C. to 50° C. and then back to 45° C., from 45° C. to 55° C. and then back to 45° C., and from 45° C. to 60° C. and then back to 45° C., respectively. FIG. 15 shows the response time of the odor manipulator 700 for different power input (0.16 W, 0.25 W, 0.3 W) to the heating electrodes for different temperature change statuses (Status A1: 45° C. to 50° C., Status A2: 45° C. to 55° C., Status A3: 45° C. to 60° C., Status B1: 50° C. to 45° C., Status B2: 55° C. to 45° C., Status B3: 60° C. to 45° C.). FIG. 16 shows the temperature fluctuation in the odor manipulator 700 for different power input (0.16 W, 0.25 W, 0.3 W) to the electrodes for different stabilized temperatures (45° C., 50° C., 55° C., 60° C.).

From FIGS. 14 to 16, it can be seen that in general, higher power input to the electrodes can generate heat more quickly to raise the temperature, which leads to a faster response time but causes more pronounced temperature fluctuation.

As shown in FIGS. 14 and 15, power input equal to or greater than 0.25 W enables significantly faster response time, for heating the electrodes to the target temperature. In contrast, the heating response time for power input 0.3 W is not exponentially reduced compared with the heating response time for power input 0.25 W. Under the same power input, the response time for cooling is comparable, as the power input is removed during the cooling period.

FIG. 16 shows a similar temperature fluctuation of about 2° C. for the different energy inputs for different stabilized temperatures (45° C., 50° C., 55° C., 60° C.). These show that the extra power input (comparing 0.3 W with 0.25 W or 0.16 W, or comparing 0.25 W with 0.16 W) does not create a significant difference in temperature fluctuation. In some cases, energy consumption may be considered when choosing power input. In this example, the power input of 0.25 W has a faster response time than the other power input 0.16, and has a similar temperature fluctuation compared with the other power input 0.16 W and 0.3 W, and a lower energy consumption than power input 0.3 W. Thus, in this example, 0.25 W is selected as the optimal power input for the electrodes of the odor manipulator 700.

Experiments are performed to investigate the effect of the power input to the electromagnetic coil (Cu coil) of the movement-mechanism-based cooling system on the corresponding electrical response of the heating temperature, the response time of temperature change, and the temperature fluctuation. In this experiment, different power input is applied to the electromagnetic coil to optimize the power input (0.24 W, 0.3 W, 0.35 W). In this example, the experimental conditions are as follows: power into heating electrodes=0.3 W, environmental temperature=19° C., wax mass=0.0025 g, and PET thickness=25 μm.

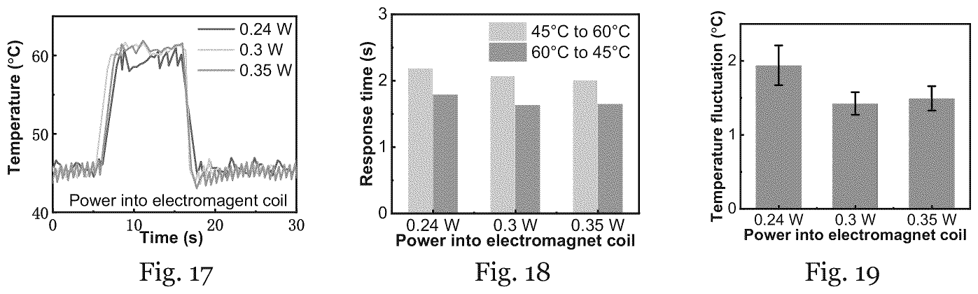

FIG. 17 shows the measured change in temperature in the odor manipulator 700 for different power input (0.24 W, 0.3 W, 0.35 W) to the electromagnetic coil, when temperature is switched from 45° C. to 60° C. and then back to 45° C. FIG. 18 shows the response time of the odor manipulator 700 for different power input (0.24 W, 0.3 W, 0.35 W) to the electromagnetic coil, when temperature is switched from 45° C. to 60° C. and then back to 45° C. FIG. 19 shows the temperature fluctuation in the odor manipulator 700 for different power input (0.24 W, 0.3 W, 0.35 W) to the electromagnetic coil.

From FIGS. 17 and 18, it can be seen that the power input to the electromagnetic coil does not significantly affect the target temperature of electrodes and the temperature response time during heating. It is worth noting that the heat transferred from the electromagnetic coil to the heating electrodes is affected by the separation between the electromagnetic coil and the heating electrodes provided by the PET, and the temperature generated from it is lower. The heating response times from 45° C. to 60° C. are similar for the different power inputs to the electromagnetic coil, while the cooling times from 60° C. to 45° C. are almost identical for the different power inputs to the electromagnetic coil. FIG. 19 shows a near temperature fluctuation range from 1.4° C. to 1.9° C. for different energy inputs to the electromagnetic coil (Cu coil).

These show that the power input on the electromagnetic coil does not create a significant difference in target temperature and temperature fluctuation in the odor manipulator 700. To balance between response time and temperature fluctuation, in one example, 0.3 W is selected to be the power applied on the electromagnetic coil to move (e.g., lift) the electrodes and promote thermal heating of odor manipulator (as heat generated by the energized electromagnetic coil reduces the thermal gradient between the electromagnetic coil and the electrodes and reduce the heat dissipation from electrodes to electromagnetic system).

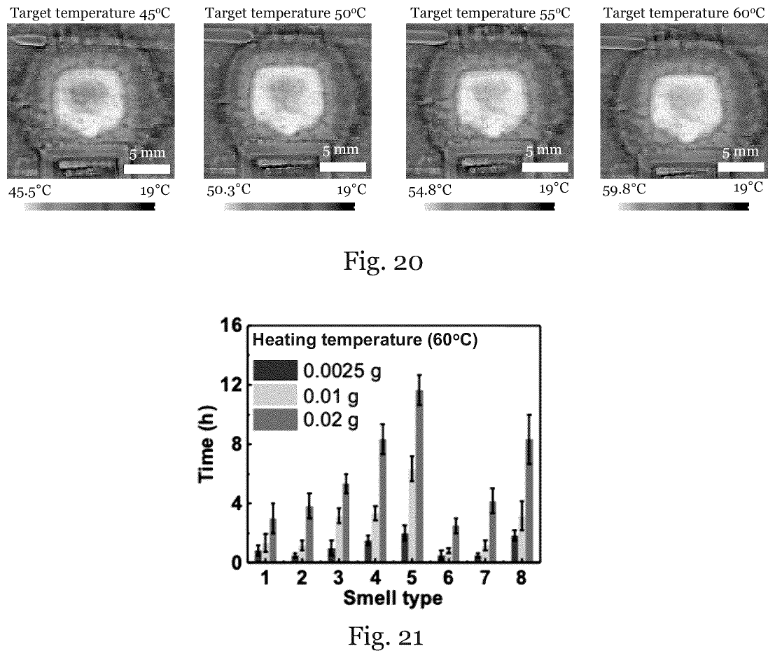

FIG. 20 shows thermal images of temperature response of the odor manipulator 700 for as the target temperature is set as different values (45° C., 50° C., 55° C., 60° C.). As shown in FIG. 20, the actual temperature is close to the set target temperature (temperature deviation less than ±0.5° C.). This indicates relatively stable operational performance of the electrodes and a controllable thermal management of electrodes. In this example, the experimental conditions are as follows: power into heating electrodes=0.25 W, power into electromagnetic coil=0.24 W. environmental temperature=19° C., wax mass=0.0025 g, and PET thickness=25 μm.

FIG. 21 shows duration of odor provided by the odor manipulator 700 for eight different odor types of different wax masses (0.0025 g, 0.01 g, 0.02 g) when the heating temperature is 60° C. The eight odor types are: 1—orange, 2—clove, 3—rosemary, 4—morning, 5—green tea, 6—clary sage, 7—mojito, and 8—lavender. It has been found that in this example increasing the mass of the medium and/or the chemical substance(s) with a 200 μm paraffin wax layer can significantly extend the duration of smell for all the eight smell types, with a remarkable 12 hours of smell for 0.02 g of type 5. In this example, the experimental conditions are as follows: power into heating electrodes=0.25 W, power into electromagnetic coil=0.24 W. environmental temperature=19° C., and PET thickness=25 μm.

The wearable electronic device in some embodiments of the invention may be flexible, made compact/small, and be arranged (e.g., programmed) to deliver specific messages to the user. The wearable electronic device can be used as a wireless information conveying device for specific individuals, such as the deaf and/or the blind. The wearable electronic device in some embodiments of the invention can be used to delivery odor for specific people, such as the deaf and/or the blind, to provide designed and programmed message. Compared with the conventional tactile devices for conveying information to the deaf and/or the blind, the wearable electronic device can convey information to the deaf and/or the blind faster and in a more straightforward way. In some embodiments, the wearable electronic device includes electro-thermal-based odor generation (OG) unit(s), and a control arrangement arranged to manage multiple channels of different primary odors in the electro-thermal-based odor generation (OG) unit(s). The control arrangement may be programmed to control the odor release time, channels, and intensities precisely and remotely, to enable mono-or stereo-olfaction. The odor can be associated with specific messages, so the wearable electronic device could realize wireless, odor-based information delivery to the deaf and/or the blind after odor training and recognition (i.e., learning by the deaf and/or the blind). In some embodiments, the wearable electronic device includes a wireless powering and communication module and an odor manipulator arrangement arranged on a flexible substrate. The odor manipulator arrangement may include one or more electro-thermal-based odor generation (OG) unit(s), which can be controlled (synchronized or desynchronized) to modulate multiple odors with spatiotemporal precision. In some embodiments, the wearable electronic device includes a flexible printed circuit board (FPCB) with surface-mounted electrical elements, which enables signal and/or data processing circuit and wireless communication. In some embodiments, the wearable electronic device can be readily worn on skin, to realize a non-intrusive and wearable device that can be mounted directly on the skin between the nose and upper lip and allowing the scent to readily reach the nostrils of the user, without substantially affecting the surrounding odor (hence without affect others in the surroundings). In some embodiments, the odor generator unit(s) can be accurately controlled to release odor in a short time (˜0.6 sec), thus enabling rapid message transfer. In some embodiments, the wearable electronic device may have a high odor generator unit(s) density (0.72/cm²), which can provide accurate and comprehensive odor profiles according to user needs and can operate relatively stably for long-term practical applications. In some embodiments, through the wireless communication module (which consumes small amount of power consumption and is low cost), odor release can be easily controlled remotely (e.g., <12 m).

The wearable electronic device in some embodiments of the invention may be used in different applications. For example, the wearable electronic device can be used to deliver information or message to user with sensory impairment such as problem with sight and/or hearing. For example, the wearable electronic device may be used as a complementary module to provide additional information (olfactory cues) to enhance learning, memory, and comprehension in education and life for the deaf and/or the blind (e.g., for identifying people, spaces, objects, and activities) as well as to affect their behavior. The wearable electronic device in some embodiments is flexible, wireless, and can provide different odor profiles, hence can directly deliver information to the deaf and/or the blind by conveying specified information through specific odors. The wearable electronic device in some embodiments includes a movement-based active cooling system that can terminate the release of odor quickly, thus avoiding excessive unwanted consumption of odorants and avoiding high concentrations of odor remaining in the surrounding area of the wearable electronic device. The wearable electronic device in some embodiments of the invention may be used as an olfactory device for extended reality (augmented reality (AR), virtual reality (VR), and mixed reality (MR)) applications. The wearable electronic device in some embodiments of the invention may be used in paramedical and medical fields (e.g., aromatherapy).

Existing olfactory devices are generally bulky, rigid (not flexible), can only provide a limited number of types of odor, may require odor cartridge replacement, etc. Existing olfactory devices are mostly only used as supplements for extended reality (augmented reality (AR), virtual reality (VR), and mixed reality (MR)) devices. Existing olfactory devices may generate odor by natural evaporation, airflow (e.g., fans), heating, or atomization, and may distribute odors by spatial diffusion (from high to low concentrations to the human olfactory organs). The wearable electronic device in some embodiments of the invention addresses one or more of these problems. The wearable electronic device in some embodiments of the invention may be configured for use by specific user, such as the deafblind. The wearable electronic device can be used to convey information by associating different odor or odor profile with different messages recognizable by the user. In some embodiments, the wearable electronic device is arranged to release odorants using an electro-thermal mechanism, which creating a broad, persistent odor, and to remove or reduce the odor by active cooling. In some embodiments, the wearable electronic device includes an electromagnetic active cooling system that operates as a switch to cool down the odor manipulation or generation unit and to facilitate solidification of the odorous phase change material (e.g., odorous paraffin wax) in a short time, which help to rapidly reduce or stop odor release. In some embodiments, odorants are not arranged in rigid replaceable cartridges, and are instead directly embedded and stored in the paraffin wax (e.g., food-grade) to carry and store the odorant, which helps to better control the odor release and removal (through the phase change of the paraffin wax). In some embodiments, the wearable electronic device includes electro-thermal-based odor generation unit(s) with multiple channels of different primary odors, and is programmed to accurately (and optionally remotely) control the odor release time, odor release channels, and odor release intensities, to enable mono-/stereo-olfaction. In some embodiments, the wearable electronic device is flexible, lightweight, and/or miniaturized, hence is beneficial for wearing on the skin of the user and may enable seamlessly attaching of the device to the human epidermis.

It will be appreciated by a person skilled in the art that variations and/or modifications may be made to the described and/or illustrated embodiments of the invention to provide other embodiments of the invention. The described/or illustrated embodiments of the invention should therefore be considered in all respects as illustrative, not restrictive. Example optional features of some embodiments of the invention are provided in the summary and the description. Some embodiments of the invention may include one or more of these optional features (some of which are not specifically illustrated in the drawings). Some embodiments of the invention may lack one or more of these optional features (some of which are not specifically illustrated in the drawings). For example, the wearable electronic device may have any number of (one or more) odor manipulation units. In wearable electronic device with multiple odor manipulation units, the units may be identical or different. For example, each odor manipulation unit may respectively have any number of (one or more) odor manipulators. In odor manipulation unit with multiple odor manipulators, the odor manipulators may be identical or different. One or more of the shape, size, form, construction, etc., of the odor manipulation unit(s) may be different from those specifically illustrated. One or more of the shape, size, form, construction, etc., of the odor manipulator(s) may be different from those specifically illustrated. For example, the wearable electronic device may lack a power module and/or a communication module, which may instead be arranged externally of the wearable electronic device. The release control mechanism of the odor manipulator can be different from those illustrated. For example, the release control mechanism may include other types of heating and/or movement mechanism, temperature detecting means, control circuit arrangements, etc.

1. A wearable electronic device for providing or affecting smell, comprising:
one or more odor manipulation units each respectively operable to provide or affect smell perceivable by a user of the wearable electronic device; and
a control circuit arrangement operably coupled with the one or more odor manipulation units for operating the one or more odor manipulation units.

2. The wearable electronic device of claim 1,
wherein the wearable electronic device further comprises a substrate; and
wherein the one or more odor manipulation units and the control circuit arrangement are supported by or mounted to the substrate.

3. The wearable electronic device of claim 2, wherein the substrate is flexible and/or elastomeric.

4. The wearable electronic device of claim 2, wherein the substrate is constructed to enable or facilitate wearing of the wearable electronic device on or near a nose of the user.

5. The wearable electronic device of claim 4, wherein the one or more odor manipulation units comprises:
a first odor manipulation unit configured to be placed closer to one nostril of the user when the wearable electronic device is worn on or near the nose of the user, and
a second odor manipulation unit configured to be placed closer to another nostril of the user when the wearable electronic device is worn on or near the nose of the user.

6. The wearable electronic device of claim 5, wherein the control circuit arrangement is arranged to independently operate the first odor manipulation unit and the second odor manipulation unit.

7. The wearable electronic device of claim 5,
wherein the first odor manipulation unit comprises two or more odor manipulators; and/or
wherein the second odor manipulation unit comprises two or more odor manipulators.

8. The wearable electronic device of claim 5,
wherein the first odor manipulation unit comprises two or more odor manipulators, each of the two or more odor manipulators of the first odor manipulation unit is respectively arranged to provide a respective odor; and/or
wherein the second odor manipulation unit comprises two or more odor manipulators, each of the two or more odor manipulators of the second odor manipulation unit is respectively arranged to provide a respective odor.

9. The wearable electronic device of claim 7,
wherein the control circuit arrangement is arranged to:
control activation and/or deactivation of each of the two or more odor manipulators of the first odor manipulation unit to affect spatial and/or temporal odor profile provided by the first odor manipulation unit; and/or
control activation and/or deactivation of each of the two or more odor manipulators of the second odor manipulation unit to affect spatial and/or temporal odor profile provided by the second odor manipulation unit.

10. The wearable electronic device of claim 9, wherein the control circuit arrangement comprises:
one or more processors; and
memory storing one or more sets of control instructions each arranged to be operated by the one or more processors to control respective activation and/or deactivation of: one or more of the odor manipulators of the first odor manipulation unit and/or one or more of the odor manipulators of the second odor manipulation unit,
wherein each of the one or more sets of control instructions is associated with a respective message recognizable by the user.

11. The wearable electronic device of claim 1,
wherein the one or more odor manipulation units comprises an odor manipulator; and
wherein the odor manipulator comprises:
a body defining:
a chamber for receiving one or more chemical substances, and
an interface through which the one or more chemical substances can be released from the body to provide or affect smell perceivable by the user.

12. The wearable electronic device of claim 11,
wherein the odor manipulator further comprises the one or more chemical substances received in the chamber, and
wherein the one or more chemical substances can (i) provide or generate an odor to affect smell perceivable by the user or (ii) react or interact with one or more substances in an environment the wearable electronic device is in to affect smell perceivable by the user.

13. The wearable electronic device of claim 11,
wherein the one or more chemical substances are included in a medium arranged to be received in the chamber, and
wherein the medium includes one or more phase change materials that can be dissolved or vaporized to facilitate release of the one or more chemical substances from the body through the interface.

14. The wearable electronic device of claim 13,
wherein the odor manipulator further comprises the medium with the one or more chemical substances received in the chamber, and
wherein the medium comprises wax.

15. The wearable electronic device of claim 13,
wherein the interface comprises one or more through-holes through which the one or more chemical substances can pass; and
wherein the body comprises a shield providing the one or more through-holes of the interface, the shield is shaped and/or sized to prevent unwanted escape or removal of non-gaseous phase of the medium from the chamber.

16. The wearable electronic device of claim 13, wherein the odor manipulator further comprises:
a release control mechanism arranged at least partly in the body and operable to control release of the one or more chemical substances from the body through the interface; and
wherein the release control mechanism comprises a heating mechanism with one or more heating elements operable to provide heat to facilitate release of the one or more chemical substances from the body through the interface.

17. The wearable electronic device of claim 16, wherein the one or more heating elements comprise one or more heating elements.

18. The wearable electronic device of claim 17,
wherein the release control mechanism further comprises a temperature sensor for providing temperature information that indicates a temperature in the chamber or a temperature of the one or more heating elements.

19. The wearable electronic device of claim 18, wherein the one or more heating elements are operable as at least part of the temperature sensor.

20. The wearable electronic device of claim 19, wherein the one or more heating elements are one or more heating electrodes.

21. The wearable electronic device of claim 18, wherein the release control mechanism further comprises a movement mechanism operable to move the one or more heating elements relative to a frame of the body to facilitate cooling of the heating mechanism and hence reduce or stop vaporization of the one or more chemical substances.

22. The wearable electronic device of claim 21, wherein the movement mechanism comprises:
a support member supporting at least the one or more heating elements; and
an actuation mechanism coupled with the support member to move the support member and hence the one or more heating elements relative to the frame of the body.

23. The wearable electronic device of claim 22,
wherein the actuation mechanism comprises a magnetic member and an electromagnetic member operable to interact to cause relative movement of the magnetic member and the electromagnetic member; and
wherein the relative movement of the magnetic member and the electromagnetic member is arranged to cause movement of the support member.

24. The wearable electronic device of claim 23, wherein movement of the support member comprises cantilever motion and/or pivoting motion.

25. The wearable electronic device of claim 18, wherein the release control mechanism further comprises:
a temperature control circuit arrangement operably connected with the one or more heating elements and the temperature sensor for controlling operation of the one or more heating elements based on temperature information provided by the temperature sensor.

26. The wearable electronic device of claim 23, wherein the release control mechanism further comprises:
a power control circuit arrangement operably connected with the electromagnetic member and the temperature sensor for controlling electric current in the electromagnetic member and hence movement of the one or more heating elements relative to the frame of the body based on temperature information provided by the temperature sensor.
